(19) United States
(12) Patent Application Publication
(10) Pub. No.: US 2012/0179202 A1
(43) Pub. Date: Jul. 12, 2012

(54) CERVICAL PILLOW FOR TREATMENT OF CERVICAL SPINE DISEASES

(76) Inventor: Loan Kim Thi Pham, HCMC (VN)

(21) Appl. No.: 13/262,635

(22) PCT Filed: Aug. 10, 2011
(86) PCT No.: PCT/IB2011/053563

§ 371 Date: Oct. 2, 2011

(30) Foreign Application Priority Data
Sep. 20, 2010 (VN) 2-2010-00198

(51) Int. Cl. A61F 5/01

(52) U.S. Cl. 606/240

(57) ABSTRACT

A cervical pillow for treatment of cervical spine diseases includes a padded member made of resilient materials such as foam, rubber, or cotton. The padded member has a rectangular flat bottom side, vertically flat left and right sides, a rounded front side, a thin back side, and a wavy top side that has a flat portion connecting to the front side, a slightly concave portion in the middle, and a very convex portion connecting to the back side. The cervical pillow further includes a nearly rectangular solid block made of high-strength materials such as plastic or compressed rubber. This solid block is embedded within the padded member along its back side and under the very convex portion of its top side.

### FIELD OF THE INVENTION

The present invention generally relates to a device for treatment of cervical spine diseases and more particularly, to a cervical pillow for treatment of cervical spine diseases.

### BACKGROUND OF THE INVENTION

Most muscles of the body relax completely when they are not being used; however, muscles in the neck have to work non-stop in order to keep the head in its place. The human neck is constantly fighting against gravity. It also has to bend, lift, turn, and twist resulting in the discs and the facet joints to degenerate. As the discs become thinner, the spaces between the vertebrae become narrower. Sustained contraction, hypertonicity, and motion restriction of the atypical and typical cervical vertebrae, C0-C2 and C2-T1, respectively, can lead to headache and neck pain, and more severely, cervical spine diseases including cervical kyphosis, straight cervical spine, and cervical herniated disc. Several attempts have been made to treat these cervical spine diseases, some of which were artificial cervical disc replacement, therapeutic drugs, and chiropractic care. However, artificial cervical disc replacement is not for everyone, especially not for those with osteoporosis, joint disease, and allergy to stainless steel. In addition, artificial cervical disc replacement requires a discectomy, which may lead to other complications such as infection, excessive bleeding, and damages to nerves, spinal cord, esophagus or vocal cord. Drugs such as acetaminophen and NSAIDs can reduce neck pain; unfortunately, they are only temporary. In addition to the drugs limited ability to treat cervical spine diseases, they cause liver and kidney damage, gastrointestinal bleeding, and ulcers. Chiropractic care seems to be a safer option of the three treatments. Chiropractors use spinal effective manipulation techniques, such as specific spinal manipulation or instrument-assisted manipulation, to treat patients. However, in the process of treating patients, there is a risk of putting excessive forces on the patients' spine that may lead to damaging it. On the other hand, if the chiropractors treat the diseases with totally safe thus much less intensive forces, the treatment period is normally very lengthy. The treatment period is even prolonged given the fact that patients can only spend one to two hours a day with the chiropractors. To treat the diseases safely and to shorten the treatment period, it becomes necessary to have a device that can replicate the safe forces applied by the chiropractors, and that is handy enough for patients to use it whenever needed in order to increase the treatment time each day, thus to shorten the treatment period.

The Olson patent (U.S. Pat. No. 5,644,809) describes a pillow for maintaining proper cervical and thoracic spinal alignment of people as they sleep. This pillow, however, does not have the functions for treating cervical spine diseases.

The Chang-Ho patent application (Publication No. WO/2009/148215) describes a pillow for protecting the cervical vertebrae by maintaining proper head and cervical vertebrae height. This pillow, however, does not have the functions for treating cervical spine diseases.

Finally, the Sang-Hyun patent application (Publication No. WO/2007/091863) describes a pillow for supporting a user's head, neck, shoulders and back. The pillow relaxes the joints of bones and muscles when the user lies on the pillow to strengthen the vertebrae, the nervous tissue and blood vessels, thereby preventing various diseases. This pillow, however, also does not have functions of treating cervical spine diseases.

### SUMMARY OF THE INVENTION

The objective of the present invention is to provide a cervical pillow that treats cervical spine diseases. These diseases include cervical kyphosis, straight cervical spine, and cervical herniated disc.

According to the present invention, a cervical pillow for treatment of cervical spine diseases includes a padded member made of resilient materials such as foam, rubber, or cotton. The padded member has a rectangular flat bottom side, vertically flat left and right sides, a rounded front side, a thin back side, and a wavy top side that has a flat portion connecting to the front side, a slightly concave portion in the middle, and a very convex portion connecting to the back side. The cervical pillow further includes a nearly rectangular solid block made of high-strength materials such as plastic or compressed rubber. This solid block is embedded within the padded member along its back side and under the very convex portion of its top side.

The very convex portion of the top side together with the solid block create an elevated firm area. When a patient lay down in a supine position in which the occipital region of his head is on the slightly concave portion of the top side and his neck is on the elevated firm area, the elevated firm area is, thanks to its very convex shape, capable of generating a strong but safe reaction force against the gravity forces from the head and body. Obviously, the reaction force is placed on his cervical spine. Given that, the elevated firm area is able to treat cervical spine diseases by gradually adjusting the straight or kyphotic cervical spine back to its natural inward curvature. With the same mechanism, the elevated firm area is also able to treat cervical herniated discs by gradually pushing the discs back into their original places.

The brief description above sets forth rather broadly the more important features of the present invention in order that the detailed description thereof that follows may be better understood, and in order that the present contributions to the art may be better appreciated. There are, of course, additional features of the invention that will be described hereinafter and which will be for the subject matter of the claims appended hereto. Before explaining the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is understood that the phraseology and terminology employed herein are for the purpose of description and should not be regarded as limiting. As such, those skilled in the art will appreciate that the conception, upon which disclosure is based, may readily be utilized as a basis for designing other structures, methods, and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

The invention will be better understood when consideration is given to the following detailed description thereof Such description makes reference to the annexed drawings wherein:

FIG. 1 is a perspective left-back view of a cervical pillow for treatment of cervical spine diseases according to the invention.

FIG. 2 is a perspective right-back view of the pillow shown in FIG. 1

FIG. 3 is a perspective right-front view of the pillow shown in FIG. 1

FIG. 4 is a perspective left-back view of the pillow shown in FIG. 1

FIG. 5 is a cross-section side view of the pillow shown in FIG. 4 taken along lines A-A

FIG. 6 is a cross-section front view of the pillow shown in FIG. 4 taken along lines B-B

FIG. 7 shows a person in a supine position lying on the pillow shown in FIG. 1

### DETAILED DESCRIPTION OF THE INVENTION

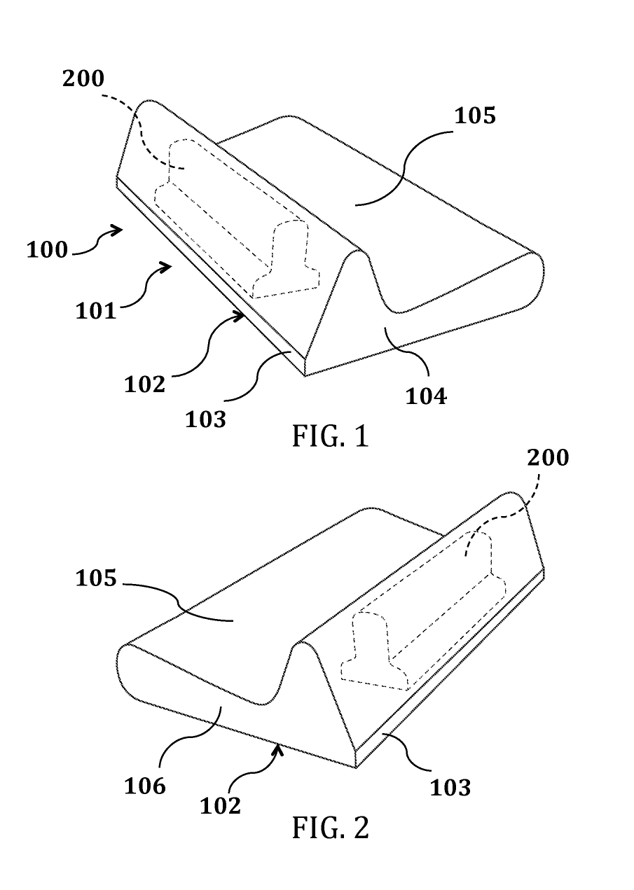

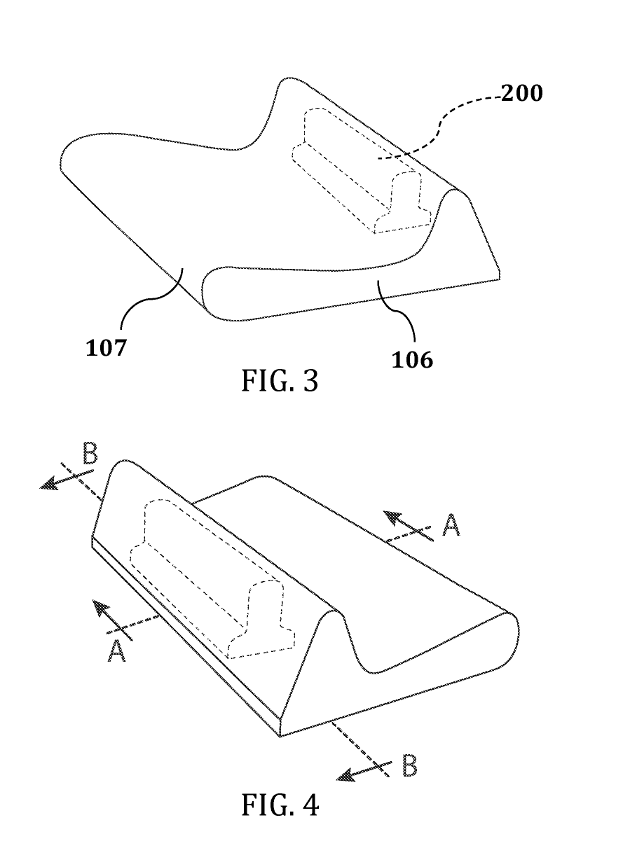

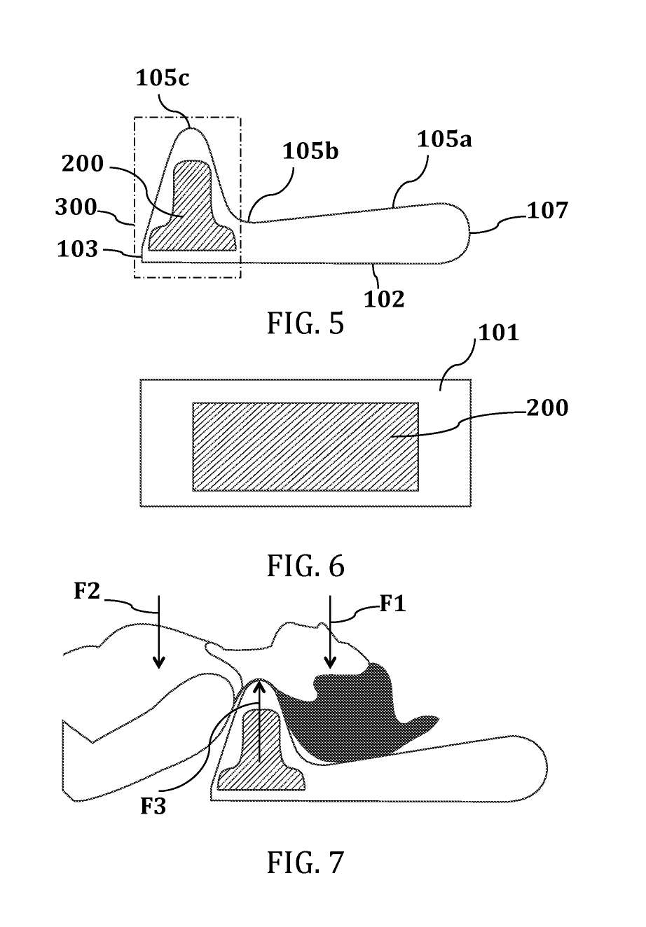

FIGS. 1, 2, and 3 show perspective views of a cervical pillow for treatment of cervical spine diseases 100, which comprises a padded member 101. The padded member 101 comprises rectangular flat bottom side 102, a vertically flat left side 104, a vertically flat right side 106, a rounded front side 107, a thin back side 103, and a wavy top side 105 that has a flat portion 105a connecting to the front side 107 and a slightly concave portion 105b in the middle and a very convex portion 105c connecting to the back side 103, and further includes a nearly rectangular solid block 200 made of high-strength materials such as solid foam, plastic, or compress rubber, inserted inside the padded member 101 along its back side 103 and under the very convex portion 105c of its top side 105.

The very convex portion 105c of the top side 105 together with the solid block 200 create an elevated firm area 300. When a patient lay down in a supine position in which the occipital region of his head is on the slightly concave portion 105b of the top side 105 and his neck is on the elevated firm area 300 as shown in FIG. 7, the elevated firm area 300 is, thanks to its very convex shape, capable of generating a strong but safe reaction force F3 against the gravity force F1 from the head and the gravity force F2 from the body. Obviously, the reaction force F3 is placed on his cervical spine. Given that, the elevated firm area is able to treat cervical spine diseases by gradually adjusting a straight or kyphotic cervical spine back to its natural inward curvature. With the same mechanism, the elevated firm area 300 is also able to treat cervical herniated discs by gradually pushing the discs back into their original places. In this case, a user needs to place his neck onto the elevated firm area in the manner that the herniated disc is right on the peak of the elevated firm area 300.

What is claimed is:

1. A cervical pillow includes a padded member made of resilient materials such as foam. The padded member, whose density is 30-40 kg/m3, has a rectangular flat bottom side, vertically flat left and right sides, a rounded front side with a height of 9 cm to 12 cm, a thin back side, and a wavy top side that has a flat portion connecting to the front side, a concave portion in the middle, and a convex portion connecting to the back side. The height of the thinnest area of the pillow is 5 to 7 cm. The cervical pillow further includes a nearly rectangular solid block made of Expanded Polystyrene (EPS) Foam whose density is from 30 to 35 kg/m3. The block includes two parts: the bell-shaped base (the lower part) and the bell-shaped body (the upper part.) The block's dimensions are as follows: The height is 115 mm, the base's width is 100 mm, the base's height is 30 mm, the body's height is 85 mm, the body's width is 45 mm, and the body's very top's width is 22 mm. The block is able to tolerate, without being broken or distorted, the gravitational force on a mass of up to 80 kg with a shape and size similar to those of a person's neck.
The pillow is designed in a way that, when a person put the back of his neck on the convex portion and his head on the concave portion of the wavy top side, his cervical lordosis will stay in a clinically normal range of 31 degrees to 40 degrees.

2. A pillow according to claim 1, wherein the nearly rectangular block is made of high-strength materials such as plastic or compressed rubber, and the padded member is made of resilient materials such as cotton or rubber.
